(19) United States
(12) Patent Application Publication
(10) Pub. No.: US 2009/0221577 A1
(43) Pub. Date: Sep. 3, 2009

(54) COMPOUNDS HAVING MORPHOLINYL AND PIPERIDINYL GROUPS FOR USE AS GLYT1 INHIBITORS

(75) Inventors: Clive Leslie Branch, Essex (GB); David John Nash, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited

(21) Appl. No.: 11/568,049

(22) PCT Filed: Apr. 15, 2005
(86) PCT No.: PCT/GB2005/001449

§ 371 Date: Oct. 18, 2006

(30) Foreign Application Priority Data
Apr. 20, 2004 (GB) 0408777.1

(51) Int. Cl. A61K 31/5377; C07D 413/12

(52) U.S. Cl. 514/235.5; 544/130

(57) ABSTRACT

The invention provides a compound of formula (I) or a salt or solvate thereof:

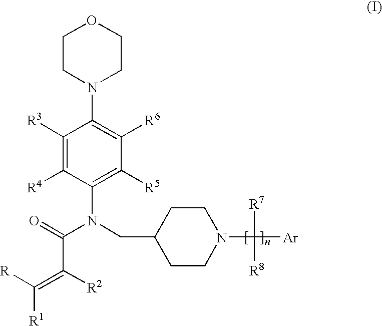

wherein R, R¹, R², R³, R⁴, R⁵, R⁶, R⁷, R⁸, n and Ar are as defined in the specification, and uses of such compounds. The compounds inhibit GlyT1 transporters and are useful in the treatment of certain neurological and neuropsychiatric disorders, including schizophrenia.

The present invention relates to glycine transporter inhibiting compounds, their use in the manufacture of medicaments for treating neurological and neuropsychiatric disorders, in particular psychoses, dementia or attention deficit disorder. The invention further comprises processes to make these compounds and pharmaceutical formulations thereof.

Molecular cloning has revealed the existence in mammalian brains of two classes of glycine transporters, termed GlyT1 and GlyT2. GlyT1 is found predominantly in the forebrain and its distribution corresponds to that of glutaminergic pathways and NMDA receptors (Smith, et al., Neuron, 8, 1992: 927-935). Molecular cloning has further revealed the existence of three variants of GlyT1, termed GlyT-Ia, GlyT-1b and GlyT-1c (Kim et al., Molecular Pharmacology, 45, 1994: 608-617), each of which displays a unique distribution in the brain and peripheral tissues. The variants arise by differential splicing and exon usage, and differ in their N-terminal regions. GlyT2, in contrast, is found predominantly in the brain stem and spinal cord, and its distribution corresponds closely to that of strychnine-sensitive glycine receptors (Liu et al., J. Biological Chemistry, 268, 1993: 22802-22808; Jursky and Nelson, J. Neurochemistry, 64, 1995: 1026-1033). Another distinguishing feature of glycine transport mediated by GlyT2 is that it is not inhibited by sarcosine as is the case for glycine transport mediated by GlyT1. These data are consistent with the view that, by regulating the synaptic levels of glycine, GlyT1 and GlyT2 selectively influence the activity of NMDA receptors and strychnine-sensitive glycine receptors, respectively.

NMDA receptors are critically involved in memory and learning (Rison and Staunton, Neurosci. Biobehav. Rev., 19 533-552 (1995); Danysz et al, Behavioral Pharmacol., 6 455-474 (1995)); and, furthermore, decreased function of NMDA-mediated neurotransmission appears to underlie, or contribute to, the symptoms of schizophrenia (Olney and Farber, Archives General Psychiatry, 52, 998-1007 (1996). Thus, agents that inhibit GlyT1 and thereby increase glycine activation of NMDA receptors can be used as novel antipsychotics and anti-dementia agents, and to treat other diseases in which cognitive processes are impaired, such as attention deficit disorders and organic brain syndromes. Conversely, over-activation of NMDA receptors has been implicated in a number of disease states, in particular the neuronal death associated with stroke and possibly neurodegenerative diseases, such as Alzheimer's disease, multi-infarct dementia, AIDS dementia, Huntington's disease, Parkinson's disease, amyotrophic lateral sclerosis or other conditions in which neuronal cell death occurs, such as stroke or head trauma. Coyle & Puttfarcken, Science, 262, 689-695 (1993); Lipton and Rosenberg, New Engl. J. of Medicine, 330, 613-622 (1993); Choi, Neuron, 1, 623-634 (1988). Thus, pharmacological agents that increase the activity of GlyT1 will result in decreased glycine-activation of NMDA receptors, which activity can be used to treat these and related disease states. Similarly, drugs that directly block the glycine site of the NMDA receptors can be used to treat these and related disease states.

Glycine transport inhibitors are already known in the art, for example as disclosed in published international patent application WO03/055478 (SmithKline Beecham).

However, there still remains the need to identify further compounds that can inhibit GlyT1 transporters, including those that inhibit GlyT1 transporters selectively over GlyT2 transporters.

It has now been found that a novel class of compounds inhibit GlyT1 transporters and are thus useful in the treatment of certain neurological and neuropsychiatric disorders, including schizophrenia.

Thus, in the first aspect, there is provided a compound of formula (I) or a salt or solvate thereof:

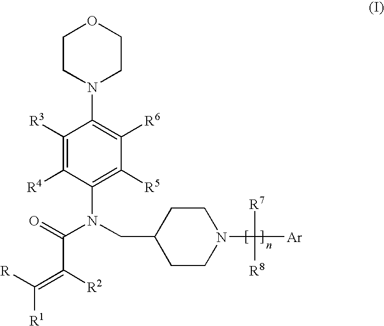

wherein:
R and R¹, together with the carbon atom to which they are attached, form optionally substituted C₃₋₈cycloalkyl or optionally substituted C₃₋₈heterocyclyl; or
R is selected from the group consisting of optionally substituted C₁₋₈alkyl, optionally substituted C₃₋₈cycloalkyl, optionally substituted C₃₋₈heterocyclyl, optionally substituted aryl and optionally substituted heteroaryl; and R¹ is selected from the group consisting of hydrogen, halogen, optionally substituted C₁₋₈alkyl and optionally substituted C₁₋₈cycloalkyl;
R² is selected from the group consisting of hydrogen, halogen, optionally substituted C₁₋₈alkyl and optionally substituted C₃₋₈cycloalkyl;
R³, R⁴, R⁵ and R⁶ are independently selected from hydrogen and halogen;
R⁷ and R⁸ are independently selected from the group consisting of hydrogen, halogen, C₁₋₄alkyl or R⁷ and R⁸ together form C₃₋₄cycloalkyl;
Ar is an optionally substituted aryl or an optionally substituted heteroaryl; and
n is 0, 1, 2 or 3.

As used herein, the term “alkyl” refers to a straight or branched alkyl in all isomeric forms. Examples of C₁₋₄alkyl include methyl, ethyl, propyl, isopropyl, butyl, isobutyl, sec-butyl and tert-butyl. Examples of C₁₋₄alkyl include, in addition to C₁₋₄alkyl, pentyl, neopentyl, sec-pentyl, n-pentyl, isopentyl, tert-pentyl, hexyl, heptyl and octyl.

As used herein, the term “cycloalkyl” refers to a non-aromatic cyclic saturated hydrocarbon ring. Examples of C₃₋₄cycloalkyl include cyclopropyl and cyclobutyl. Examples of C₃₋₈cycloalkyl include, in addition, cyclopentyl, cyclohexyl, cycloheptyl and cyclooctanyl.

As used herein, the term “C₃₋₈heterocyclyl” refers to a C₃₋₈cycloalkyl group wherein one to three of the carbon atoms are replaced by heteroatom(s) independently selected from N, O and S. Examples include aziridinyl, oxetanyl, oxiranyl, azetidinyl, pyrrolidinyl, imidazolidinyl, pyrazolidinyl, tetrahydrofuranyl, dioxolanyl, piperidinyl, piperazinyl, morpholinyl, thiomorpholinyl, tetrahydrothienyl, tetrahydropyranyl, dioxanyl, dithianyl, azepanyl and octahydroazocinyl. As used herein, the term “C₄₋₈heterocyclyl” refers to a C₄₋₈cycloalkyl group wherein one to three of the carbon atoms are replaced by heteroatom(s) independently selected from N, O and S. Examples include azetidinyl, pyrrolidinyl, imidazolidinyl, pyrazolidinyl, tetrahydrofuranyl, dioxolanyl, piperidinyl, piperazinyl, morpholinyl, thiomorpholinyl, tetrahydrothienyl, dioxanyl, dithianyl, azepanyl and octahydroazocinyl.

As used herein, the term “aryl” refers to a 5- or 6-membered monocyclic aromatic group or a 8- to 11-membered bicyclic aromatic group. Examples include phenyl, indenyl, azulenyl and naphthyl.

As used herein, the term “heteroaryl” refers to a 5- or 6-membered monocyclic aromatic group wherein one, two, three or four carbon atoms are replaced by a heteroatom independently selected from N, O and S, or a 8- to 11-membered bicyclic aromatic group wherein one to four carbon atoms are replaced by a heteroatom independently selected from N, O and S, and wherein one of the rings may be partially or fully saturated. Examples of 5- or 6-membered monocyclic heteroaryls include furanyl, thienyl, pyrrolyl, oxazolyl, thiazolyl, imidazolyl, oxadiazolyl, thiadiazolyl, pyridinyl, triazolyl, triazinyl, pyridazinyl, pyrimidinyl, isothiazolyl, isoxazolyl, pyrazinyl, pyrazolyl and pyrimidinyl; examples of 8 to 11-membered bicyclic heteroaryls wherein both rings are aromatic include quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridinyl, pyridopyrimidinyl and isoquinolinyl. Examples of 8- to 11-membered bicyclic heteroaryls wherein one of the rings is partially or fully saturated include dihydrobenzofuranyl, indanyl, tetrahydronaphthyl, indolinyl, isoindolinyl, tetrahydroisoquinolinyl, tetrahydroquinolinyl, benzoxazinyl and benzoazepinyl.

As used herein, the terms “halogen” and its abbreviation “hal” refer to fluorine, chlorine, bromine, or iodine.

As used herein, the term “salt” refers to any salt of a compound according to the present invention prepared from an inorganic or organic acid or base, quaternary ammonium salts and internally formed salts. Physiologically acceptable salts are particularly suitable for medical applications because of their greater aqueous solubility relative to the parent compounds. Such salts must clearly have a physiologically acceptable anion or cation. Suitably physiologically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example benzenesulfonic and p-toluenesulfonic, acids; base addition salts formed with alkali metals and alkaline earth metals and organic bases such as N,N-dibenzylethylenediamine, chloroprocaine, choline, diethanolamine, ethylenediamine, meglumaine (N-methylglucamine), lysine and procaine; and internally formed salts. Salts having a non-physiologically acceptable anion or cation are within the scope of the invention as useful intermediates for the preparation of physiologically acceptable salts and/or for use in non-therapeutic, for example, in vitro, situations.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include water, ethanol and acetic acid. Most preferably the solvent used is water.

As used herein, the term “optionally substituted” refers to substitution by one or more groups selected from:
halogen, hydroxy, oxo, cyano, nitro, C₁₋₆alkyl, C₁₋₄alkoxy, halo_{C1-4}alkyl, haloC₁₋₄alkoxy, arylC₁₋₄alkoxy, C₁₋₄alkylthio, hydroxyC₁₋₄alkyl, C₁₋₄alkoxyC₁₋₄alkyl, C₃₋₆cycloalkyl, C₃₋₆cycloalkylC₁₋₄alkoxy, C₁₋₄alkanoyl, C₁₋₄alkoxycarbonyl, C₁₋₄alkylsulfonyl, C₁₋₄alkylsulfonyloxy, C₁₋₄alkylsulfonylC₁₋₄alkyl, arylsulfonyl, arylsulfonyloxy, arylsulfonylC₁₋₄alkyl, C₁₋₄alkylsulfonamido, C₁₋₄alkylamido, C₁₋₄alkylsulfonamidoC₁₋₄alkyl, C₁₋₄alkylamidoC₁₋₄alkyl, arylsulfonamido, arylcarboxamido, arylsulfonamidoC₁₋₄alkyl, arylcarboxamidoC₁₋₄alkyl, aroyl, aroylC₁₋₄alkyl, arylC₁₋₄alkanoyl, C₁₋₄acyl, aryl, arylC₁₋₄alkyl, C₁₋₄alkylaminoC₁₋₄alkyl, a group R⁹R¹⁰N—, R⁹OCO(CH₂)ₘ, R⁹CON(R¹⁰)(CH₂)ₘ, R⁹R¹⁰NCO(CH₂)ₘ, R⁹R¹⁰NSO₂(CH₂)ₘ or R⁹SO₂NR¹⁰(CH₂)ₘ (where each of R⁹ and R¹⁰ is independently selected from hydrogen or C₁₋₄alkyl, or where appropriate R⁹R¹⁰ forms part of a C₃₋₆azacyloalkane or C₃₋₆(2-oxo)azacycloalkane ring and m is 0, 1, 2, 3 or 4), a group R⁹R¹⁰N(CH₂)p- or R⁹R¹⁰N(CH₂)pO— (wherein p is 1, 2, 3 or 4); wherein when the substituent is R⁹R¹⁰N(CH₂)p- or R⁹R¹⁰N(CH₂)pO, R⁹ with at least one CH₂ of the (CH₂)p portion of the group may also form a C₃₋₆azacycloalkane and R¹⁰ may be hydrogen, C₁₋₄alkyl or with the nitrogen to which it is attached, form a second C₃₋₆azacycloalkane fused to the first C₃₋₆azacycloalkane. Furthermore, when R is an optionally substituted C₃₋₆cycloalkyl or an optionally substituted C₃₋₈heterocyclyl, the optionally substituted cycloalkyl or heterocyclyl group may be additionally optionally bridged by a C₁₋₃alkylene group.

Where there is more than one substituent, the substituents may be different or the same. If substituent(s) is/are present, preferably the number of substituent(s) is 1, 2, 3 or 4.

In one embodiment, R and R¹ together form optionally substituted C₃₋₈cycloalkyl or optionally substituted C₃₋₈heterocyclyl. For example, R and R¹ may form tetrahydropyranyl, cyclohexyl, piperidinyl (which would form tetrahydropyranylidene, cyclohexylidene and piperidinylidene respectively with the double bond).

In another embodiment, R is aryl, such as phenyl and R¹ is as defined above.

In one embodiment, R¹ and R² are both hydrogen.

In one embodiment, R³, R⁴ and R⁵ are hydrogen and R⁶ is fluorine.

In one embodiment, Ar is an optionally substituted aryl, such as phenyl optionally substituted by one or two groups selected from C₁₋₄alkyl, CF₃, halogen and C₃₋₆cycloalkyl.

In one embodiment, n is 1.

In one embodiment, R⁷ and R⁸ are both hydrogen.

In one embodiment, the present invention provides a compound of formula (Ia) or a salt or solvate thereof:

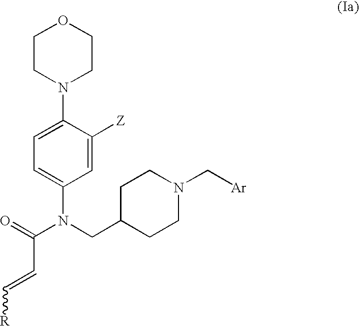

wherein
R is selected from the group consisting of C₁₋₈alkyl, C₃₋₈cycloalkyl, C₃₋₈heterocyclyl, aryl or heteroaryl, each of which is optionally substituted by one to three groups selected from the group consisting of halogen, oxo, cyano, C₁₋₆alkyl, C₁₋₄alkoxy, haloC₁₋₄alkyl, haloC₁₋₄alkoxy, arylC₁₋₄alkoxy, C₁₋₄alkylthio, C₁₋₄alkoxyC₁₋₄alkyl, C₃₋₆cycloalkyl, C₃₋₆cycloalkylC₁₋₄alkoxy, C₁₋₄alkanoyl, C₁₋₄alkylsulfonyl, C₁₋₄alkylsulfonylC₁₋₄alkyl, arylsulfonyl, arylsulfonylC₁₋₄alkyl, C₁₋₄alkylamido, C₁₋₄alkylsulfonamidoC₁₋₄alkyl, aroyl, aroylC₁₋₄alkyl, C₁₋₄acyl, aryl, arylC₁₋₄alkyl, C₁₋₄alkylaminoC₁₋₄alkyl, a group R⁹R¹⁰N—, R⁹CON(R¹⁰)(CH₂)ₘ or R⁹R¹⁰NCO(CH₂)ₘ (where each of R⁹ and R¹⁰ is independently selected from hydrogen or C₁₋₄alkyl, or where appropriate R⁹R¹⁰ forms part of a C₃₋₆azacycloalkane ring and m is 0, 1, 2, 3 or 4);
Z is hydrogen, fluorine or chlorine; and
Ar is phenyl or heteroaryl, each of which is optionally substituted by one to three groups selected from the group consisting of halogen, oxo, cyano, C₁₋₆alkyl, C₁₋₄alkoxy, haloC₁₋₄alkyl, haloC₁₋₄alkoxy, arylC₁₋₄alkoxy, C₁₋₄alkylthio, C₁₋₄alkoxyC₁₋₄alkyl, C₃₋₆cycloalkyl, C₃₋₆cycloalkylC₁₋₄alkoxy, C₁₋₄alkanoyl, C₁₋₄alkylsulfonyl, C₁₋₄alkylsulfonylC₁₋₄alkyl, arylsulfonyl, arylsulfonylC₁₋₄alkyl, C₁₋₄alkylamido, C₁₋₄alkylsulfonamidoC₁₋₄alkyl, aroyl, aroylC₁₋₄alkyl, C₁₋₄acyl, aryl, arylC₁₋₄alkyl, C₁₋₄alkylaminoC₁₋₄alkyl, a group R⁹R¹⁰N—, R⁹CON(R¹⁰)(CH₂)ₘ or R⁹R¹⁰NCO(CH₂)ₘ (where each of R⁹ and R¹⁰ is independently selected from hydrogen or C₁₋₄alkyl, or where appropriate R⁹R¹⁰ forms part of a C₃₋₆azacyloalkane ring and m is 0, 1, 2, 3 or 4).

All embodiments and features of compounds of formula (I) apply to compounds of formula (Ia).

It is to be understood that the various aspects of preferred embodiments can each, where not inappropriate, be combined with aspects of other preferred embodiments.

Examples of compounds of the invention include, (2E)-N-({1-[(4-ethylphenyl)methyl]-4-piperidinyl}methyl)-N-[3-fluoro-4-(4-morpholinyl)phenyl]-3-phenyl-2-propenamide, and salts and solvates thereof.

The compounds of formula (I) may have the ability to crystallise in more than one form. This is a characteristic known as polymorphism, and it is understood that such polymorphic forms (“polymorphs”) are within the scope of formula (I). Polymorphism generally can occur as a response to changes in temperature or pressure or both and can also result from variations in the crystallisation process. Polymorphs can be distinguished by various physical characteristics known in the art such as x-ray diffraction patterns, solubility, and melting point.

Certain of the compounds described herein may exist in stereoisomeric forms (i.e. they may contain one or more asymmetric carbon atoms or may exhibit cis-trans isomerism). The individual stereoisomers (enantiomers and diastereolsomers) and mixtures of these are included within the scope of the present invention. Thus, each form of formula (I) as shown below, as well as mixtures thereof, are within the scope of the present invention.

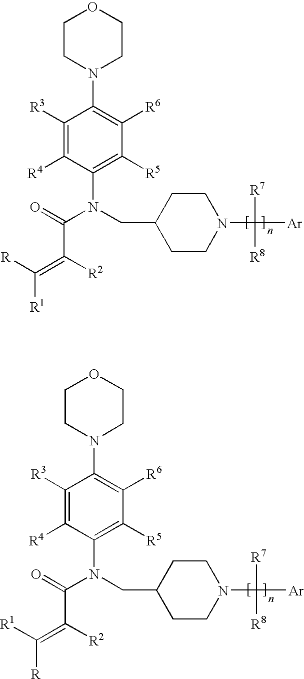

Likewise, it is understood that compounds of formula (I) may exist in tautomeric forms other than that shown in the formula and these are also included within the scope of the present invention.

As referred to above, individual enantiomers of compounds of formula (I) may be prepared and an indication of the preferred stereochemistry for such enantiomers has been given. In a preferred embodiment, an optically pure enantiomer is desired. The term “optically pure enantiomer” means that the compound contains greater than about 90% of the desired isomer by weight, preferably greater than about 95% of the desired isomer by weight, and most preferably greater than about 99% of the desired isomer by weight, said weight percent based upon the total weight of the isomer(s) of the compound.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

Compounds of general formula (I) may be prepared by methods disclosed in the documents hereinbefore referred to and by methods known in the art of organic synthesis as set forth in part by the following synthesis schemes. It is also recognised that in all of the schemes described below, it is well understood that protecting groups for sensitive or reactive groups are employed where necessary in accordance with general principles of chemistry. Protecting groups are manipulated according to standard methods of organic synthesis (T. W. Greene and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I). Those skilled in the art will recognise if a stereocentre exists in compounds of formula (I). Accordingly, the present invention includes both possible stereoisomers and includes not only racemic compounds but the individual enantiomers as well. Where the stereochemistry is indicated as being variable at certain positions, a mixture of stereoisomers may be obtained, this mixture having been separated where indicated. Stereoisomers may be separated by high-performance liquid chromatography or other appropriate means. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

Typical reaction routes for the preparation of a compound of formula (I) as hereinbefore defined, are shown in Schemes 1 and 2.

Starting materials of general structure (2) and (3) and reagents (7), (8), (9) and (13) are known in the literature or can be prepared using methods known in the art.

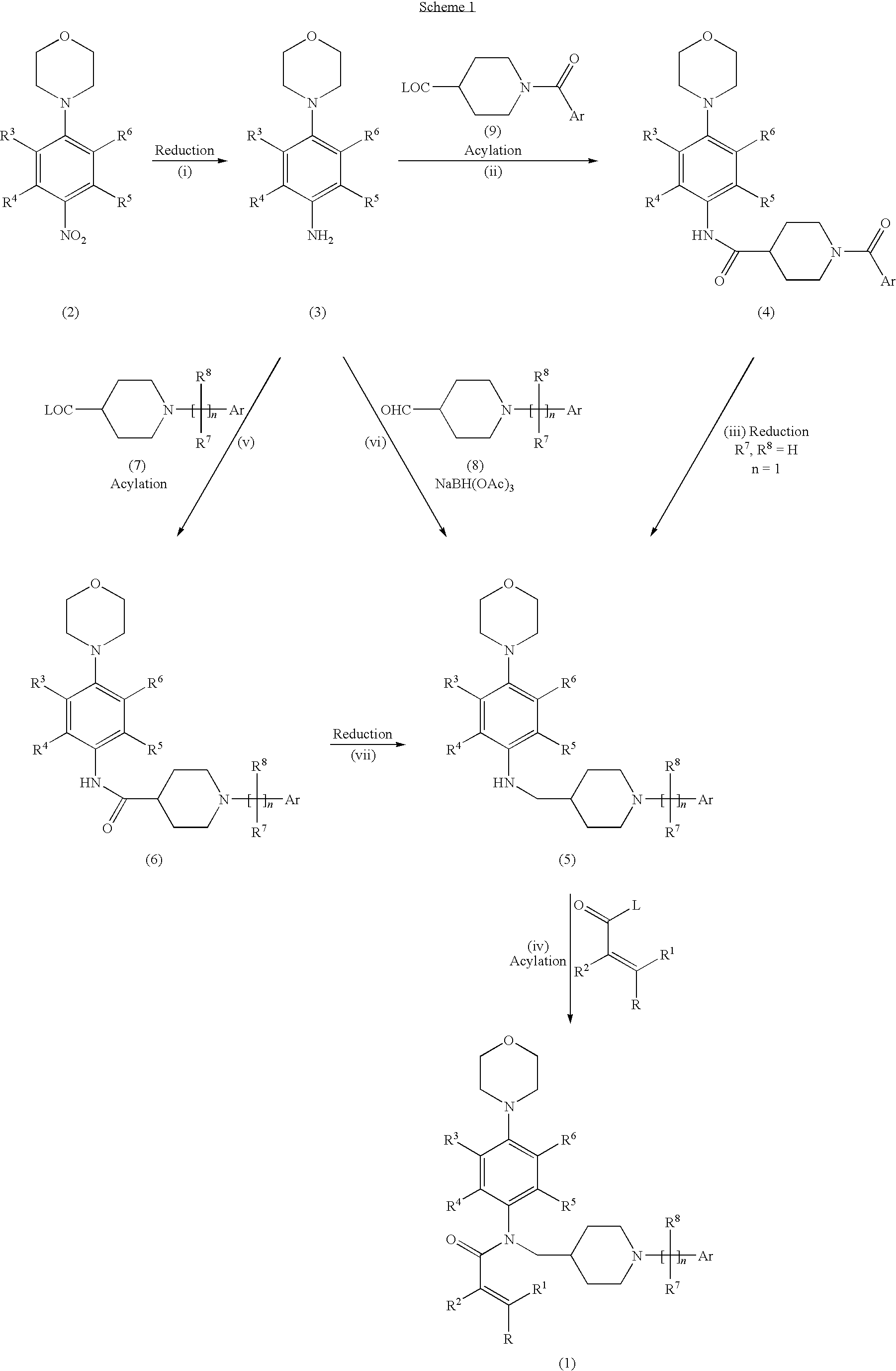

wherein R and R¹-R⁸, n and Ar are as defined above for (1) and L is a leaving group.

Examples of leaving groups include halogen, hydroxy, OC(═O)alkyl, OC(═O)O-alkyl and OSO₂Me. Preferably L is halogen and acylation in steps (ii) (iv) and (v) may be carried out in an inert solvent such as dichloromethane, in the presence of a base such as triethylamine. When L represents hydroxy, the reaction takes place in an inert solvent such as dichloromethane in the presence of a diimide reagent such as [O-(7-azabenzotriazol-1-yl)-1,1,3,3-tetramethyluronium hexafluoro phosphate].

Reduction step (i) can be accomplished using known methods, such as catalytic hydrogenolysis in an inert solvent (e.g. using palladium on charcoal in a lower alcohol or ethyl acetate), catalytic transfer hydrogenolysis (e.g. using palladium black and formic acid in methanol) or chemical reduction (e.g. iron and acetic acid).

Reduction step (iii) and (vii) can be achieved using standard methods such as reduction with lithium aluminium hydride in an inert solvent such as tetrahydrofuran. The reductive amination step (vi) can be carried out using known methods. e.g reaction of (3) with an aldehyde (8) in the presence of a reducing agent such as sodium triacetoxyborohydride in an inert solvent such as 1,2-dichloroethane or dichloromethane.

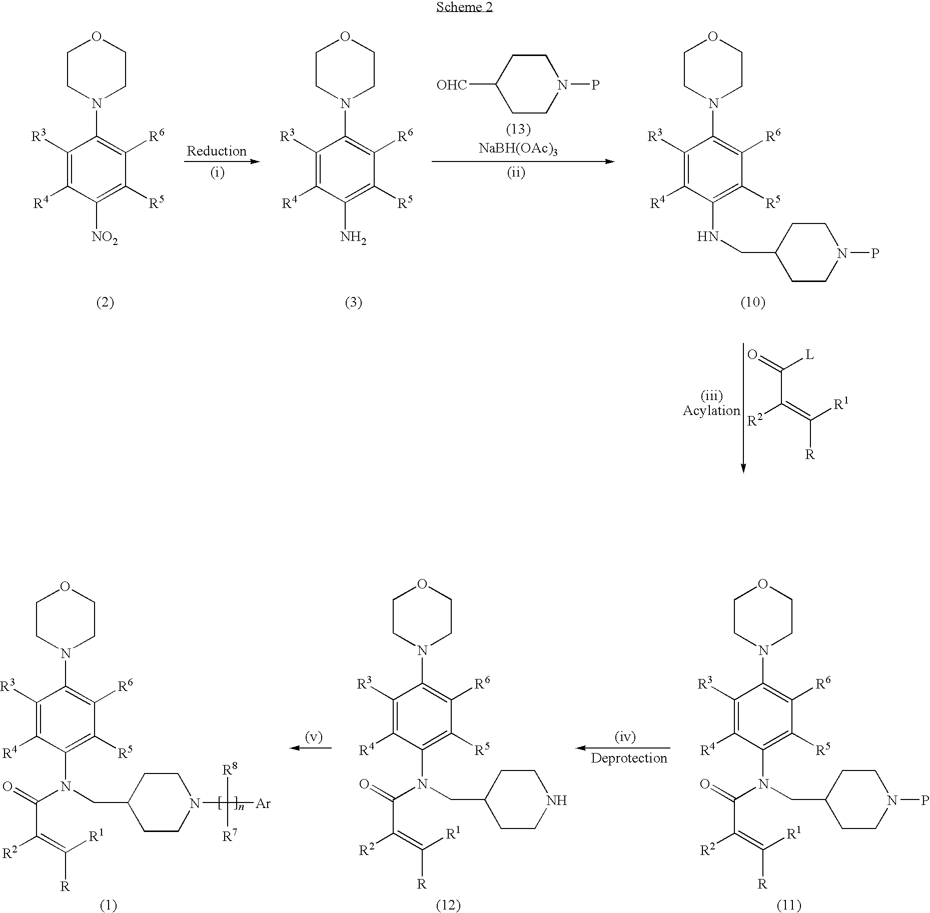

wherein R, R¹-R⁸, n and Ar are as defined above for (1), L is a leaving group as defined for scheme 1 and P is a protecting group.

Examples of protecting groups P include t-butyloxycarbonyl, trifluoroacetyl, benzyloxycarbonyl and optionally substituted benzyl. Deprotection conditions will depend on the particular protecting group; for the groups mentioned above these are respectively, acid (e.g. trifluoroacetic acid in dichloromethane), base (e.g. potassium carbonate in a solvent such as aqueous methanol) and catalytic hydrogenolysis in an inert solvent (e.g. using palladium on charcoal in a lower alcohol or ethyl acetate). Within the scope there is provision for protecting group interchange.

Reduction step (i), reductive amination step (ii) and acylation step (iii) can be achieved as described for scheme 1. Conversion of amine (12) into compound (I) can be accomplished by known methods such as reductive amination with an appropriate ketone (R⁷ or R⁸═H) or aldehyde (R⁷═R⁸═H), or through N-alkylation with an alkylating agent in the presence of base, such as potassium carbonate, and in an inert solvent such as dimethylformamide. Alternatively, alkylation can be achieved with a suitable alcohol under Mitsunobu conditions ie in an inert solvent such as dichloromethane or tetrahydrofuran, in the presence of a phosphine reagent such as triphenylphosphine or tributylphosphine, and an azodicarbonyl reagent such as diethyl azodicarboxylate, diisopropylazodicarboxylate, or 1,1′-azodicarbonyldipiperidine.

Accordingly, in a second aspect, the present invention provides a method of preparing a compound of formula (I), comprising the step of:

(a) reacting a compound of formula (II):

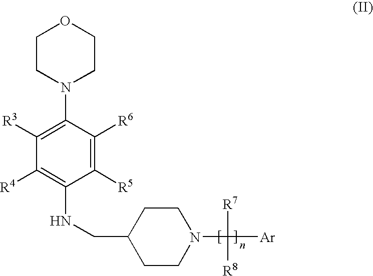

wherein R³ to R⁸, n and Ar are as defined in formula (I), with a compound of formula (III):

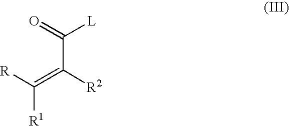

wherein R, R¹ and R² are as defined for formula (I) and L is a leaving group; or
(b) reacting a compound of formula (IV):

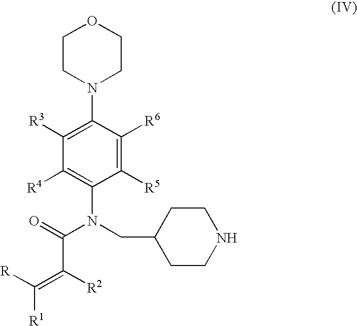

wherein R and R¹ to R⁶ are as defined for formula (I), with a compound of formula (V):

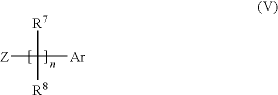

wherein R⁷, R⁸, n and Ar are as defined for formula (I) and Z is a leaving group such as halogen, hydroxy or trifluoromethanesulfonyloxy; or
(c) for a compound of formula (I) wherein n is 1, 2 or 3, reacting a compound of formula (IV) as defined above with a compound of formula (VI):

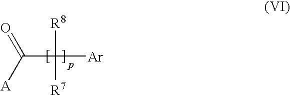

wherein R⁷, R⁸ and Ar are as defined for formula (I), p is n minus one, and A is R⁷ or R⁸;
and then optionally for step (a), step (b) or step (c):
removing any protecting groups and/or
converting a compound of formula (I) into another compound of formula (I) and/or
forming a salt or solvate.

Compounds of formula (I) can be converted into further compounds of formula (I) using standard techniques. For example, and by way of illustration rather than limitation, possible conversion reactions include acylation with an appropriate acylating agent such as acetyl chloride, alkylation using an appropriate alkylating reagent such as methyl iodide, and sulfonylation using a sulfonylating agent such as methanesulfonic anhydride.

Pharmaceutically acceptable salts may be prepared conventionally by reaction with the appropriate acid or acid derivative.

The compounds of the present invention inhibit the GlyT1 transporter. The compounds may selectively inhibit the GlyT1 transporter over the GlyT2 transporter.

Such compounds would be suitable for the treatment of certain neurological and neuropsychiatric disorders. As used herein, the terms “treatment” and “treating” refer to the alleviation and/or cure of established symptoms as well as prophylaxis.

The affinities of the compounds of this invention for the GlyT1 transporter can be determined by the following assay:

HEK293 cells expressing the Glycine (Type 1) transporter were grown in cell medium (DMEM/NUT mix F12) containing 2 mM L-Glutamine, 0.8 mg/mL G418 and 10% heat inactivated fetal calf serum (Gibco BRL) at 37° C. in 5% CO₂. Cells grown to 70-80% confluency in T175 flasks were harvested and resuspended at 1.6×10⁶ cells/ml in assay buffer [NaCl (140 mM), KCl (5.4 mM), CaCl₂ (1.8 mM), MgSO₄ (0.8 mM), HEPES (20 mM), glucose (5 mM) and alanine (5 mM), pH 7.4]. An equal volume of Leadseeker™ SPA beads (12.5 mg/ml suspended in assay buffer) was added to the cells and 25 μL of the cell/bead suspension transferred to each well of a 384-well white solid bottom plate (20,000 cells/well) that contained 14 μL of assay buffer. Compounds were prepared as 10 mM stocks in DMSO. Two-fold serial dilutions of the compounds were made in DMSO from a top concentration of 5 mM. 1 μL of compound at each concentration was added to the assay plate using 384-well parallel dispensing. Substrate (10 μL) was added to each well [1:40 dilution of [³H]-glycine in assay buffer containing 5 mM glycine). Final DMSO concentration=2%. Data was collected using a PerkinElmer Viewlux as 5 minute exposures. IC₅₀ values were determined using Grafit.

The following alternative assay may also be used:

HEK293 cells expressing the Glycine (Type 1) transporter were grown in cell culture medium [DMEM/NUT mix F12 containing 2 mM L-Glutamine, 0.8 mg/mL G418 and 10% heat inactivated fetal calf serum] at 37° C. and 5% CO₂. Cells grown to 70-80% confluency in T175 flasks were harvested and resuspended at 1.6×10⁶ cells/mL in assay buffer [140 mM NaCl, 5.4 mM KCl, 1.8 mM CaCl₂, 0.8 mM MgSO₄, 20 mM HEPES, 5 mM glucose and 5 mM alanine, pH 7.4]. Compounds were serially diluted 2-fold in DMSO from a top concentration of 10 mM with each compound giving a 16 data point dose-response. 250 nL of compound at each concentration was added to the assay plate. An equal volume of Leadseeker™ WGA SPA beads (12.5 mg/ml suspended in assay buffer) was added to the cell suspension and 12.5 μL of the cell/bead suspension transferred to each well of a 384-well white solid bottom plate (10,000 cells/well) containing 250 nL of test compounds. Substrate (12.5 μL) was added to each well [1:100 dilution of [³H]-glycine stock in assay buffer containing 2 mM glycine). Final DMSO concentration was 1% v/v. Data was collected using a Perkin Elmer Viewlux. IC₅₀ values were determined using Grafit.

Compounds are considered to have activity at the GlyT1 transporter if they have a plC₅₀ of 4.8 or above, or greater than 30% inhibition at a concentration of 10 μM. The example compounds below were found to have a pIC₅₀ at the GlyT1 transporter of greater than 6.0. Preferred compounds of the invention were found to have a pIC₅₀ at the GlyT1 transporter of greater than 6.0.

Accordingly, in a further aspect of the invention, there is provided a compound of formula (I) as hereinbefore described or a salt or solvate thereof, for use in therapy.

In another aspect of the invention, there is provided a compound of formula (I) as hereinbefore described or a salt or solvate thereof, for use in the treatment of a disorder mediated by GlyT1.

As used herein, the term La disorder mediated by GlyT1″ refers to a disorder that may be treated by the administration of a medicament that alters the activity of the GlyT1 transporter. As hereinbefore described, the action of GlyT1 transporters affects the local concentration of glycine around NMDA receptors. As a certain amount of glycine is needed for the efficient functioning of NMDA receptors, any change to that local concentration can affect NMDA-mediated neurotransmission. As hereinbefore described, changes in NMDA-mediated neurotransmission have been implicated in certain neuropsychiatric disorders such as dementia, depression and psychoses, for example schizophrenia, and learning and memory disorders, for example attention deficit disorders and autism. Thus, alterations in the activity of the GlyT1 transporter are expected to influence such disorders.

The disorders mediated by GlyT1 referred to herein include neurological and neuropsychiatric disorders, including psychoses such as schizophrenia, dementia and other forms of impaired cognition such as attention deficit disorders and organic brain syndromes. Other neuropsychiatric disorders include drug-induced (phencyclidine, ketamine and other dissociative anesthetics, amphetamine and other psychostimulants and cocaine) psychosis, psychosis associated with affective disorders, brief reactive psychosis, schizoaffective psychosis, and psychosis NOS, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), and NMDA receptor-related disorders such as autism, depression, benign forgetfulness, childhood learning disorders and closed head injury.

In another aspect of the invention, there is provided a method of treating a mammal, including a human, suffering from or susceptible to a disorder mediated by GlyT1, which comprises administering an effective amount of a compound of formula (I) as hereinbefore defined or a salt or solvate thereof.

In another aspect of the invention, there is provided use of a compound of formula (I) as hereinbefore defined or a salt or solvate thereof in the preparation of a medicament for the treatment of a disorder mediated by GlyT1.

Preferably, the disorder mediated by GlyT1 to be treated by the use or method as hereinbefore described is a psychosis, including schizophrenia, dementia and attention deficit disorders, particularly schizophrenia.

As used herein, the term “effective amount” means that amount of a drug or pharmaceutical agent that will elicit the biological or medical response of a tissue, system, animal or human that is being sought, for instance, by a researcher or clinician.

Compounds for use according to the invention may be administered as the raw material but the active ingredients are preferably provided in the form of pharmaceutical compositions.

Accordingly, in a further aspect of the invention, there is provided a pharmaceutical composition comprising a compound of formula (I) as hereinbefore described or a salt or solvate thereof, and at least one pharmaceutically acceptable carrier, diluent or excipient.

These pharmaceutical compositions may be used in the treatment of clinical conditions for which a GlyT1 inhibitor is indicated such as, for example, schizophrenia. The carrier must be pharmaceutically acceptable to the recipient and must be compatible with, i.e. not have a deleterious effect upon, the other ingredients in the composition. The carrier may be a solid or a liquid and is preferably formulated with at least one compound of formula (I) or a salt or solvate thereof as a unit dose formulation. If desired, other physiologically active ingredients may also be incorporated in the pharmaceutical compositions of the invention.

Within the context of the present invention, the terms used herein are classified in the Diagnostic and Statistical Manual of Mental Disorders, 4^{th} Edition, published by the American Psychiatric Association (DSM-IV) and/or the International Classification of Diseases, 10^{th} Edition (ICD-10). The various subtypes of the disorders mentioned herein are contemplated as part of the present invention. Numbers in brackets after the listed diseases below refer to the classification code in DSM-IV.

In particular, the compounds of formula (I) are of use in the treatment of schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

The compounds of formula (I) are also of use in the treatment of mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

The compounds of formula (I) are also of use in the treatment of anxiety disorders including Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder and Anxiety Disorder Not Otherwise Specified (300.00).

The compounds of formula (I) are also of use in the treatment of substance-related disorders including Substance Use Disorders such as Substance Dependence and Substance Abuse; Substance-Induced Disorders such as Substance Intoxication, Substance Withdrawal, Substance-induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-induced Persisting Dementia, Alcohol-induced Persisting Amnestic Disorder, Alcohol-induced Psychotic Disorder, Alcohol-Induced Mood Disorder, Alcohol-Induced Anxiety Disorder, Alcohol-Induced Sexual Dysfunction, Alcohol-induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-induced Psychotic Disorder, Cocaine-Induced Mood Disorder, Cocaine-Induced Anxiety Disorder, Cocaine-Induced Sexual Dysfunction, Cocaine-induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-induced Psychotic Disorder, Hallucinogen-induced Mood Disorder, Hallucinogen-induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-induced Persisting Dementia, Inhalant-Induced Psychotic Disorder, Inhalant-induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

The compounds of formula (I) are also of use in the treatment of sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type.

The compounds of formula (I) are also of use in the treatment of eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50).

The compounds of formula (I) are also of use in the treatment of Autistic Disorder (299.00); Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

The compounds of formula (I) are also of use in the treatment of Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9).

The compounds of Formula (I) are also of use in the enhancement of cognition including the treatment of cognition impairment in other diseases such as schizophrenia, bipolar disorder, depression, other psychiatric disorders and psychotic conditions associated with cognitive impairment. Within the context of the present invention, the term cognitive impairment includes for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypotiroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

The compounds of formula (I) are also of use in the treatment of sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

The invention also provides a compound of formula (I) as hereinbefore described or a pharmaceutically acceptable salt or solvate thereof for use in the treatment of schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders.

The invention also provides a compound of formula (I) as hereinbefore described or a pharmaceutically acceptable salt or solvate thereof for use in the treatment of psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

In another aspect of the invention, there is provided a method of treating a mammal, including a human, suffering from or susceptible to a disorder mediated by GlyT1, which comprises administering an effective amount of a compound of formula (I) as hereinbefore defined or a salt or solvate thereof.

The invention also provides a method of treating schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders which comprises administering to a mammal in need thereof an effective amount of a compound of formula (I) as hereinbefore described or a pharmaceutically acceptable salt or solvate thereof.

The invention also provides a method of treating psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders which comprises administering to a mammal in need thereof an effective amount of a compound of formula (I) as hereinbefore described or a pharmaceutically acceptable salt or solvate thereof.

In another aspect of the invention, there is provided use of a compound of formula (I) as hereinbefore defined or a salt or solvate thereof in the preparation of a medicament for the treatment of a disorder mediated by GlyT1.

Preferably, the disorder mediated by GlyT1 to be treated by the use or method as hereinbefore described is a psychosis, including schizophrenia, dementia and attention deficit disorders, particularly schizophrenia.

The invention also provides the use of a compound of formula (I) as hereinbefore described or a pharmaceutically acceptable salt or solvate thereof in the manufacture of a medicament for the treatment of schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders.

The invention also provides the use of a compound of formula (I) as hereinbefore described or a pharmaceutically acceptable salt or solvate thereof in the manufacture of a medicament for the treatment of psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

As used herein, the term “effective amount” means that amount of a drug or pharmaceutical agent that will elicit the biological or medical response of a tissue, system, animal or human that is being sought, for instance, by a researcher or clinician.

Compounds for use according to the invention may be administered as the raw material but the active ingredients are preferably provided in the form of pharmaceutical compositions.

Accordingly, in a further aspect of the invention, there is provided a pharmaceutical composition comprising a compound of formula (I) as hereinbefore described or a salt or solvate thereof, and at least one pharmaceutically acceptable carrier, diluent or excipient. These pharmaceutical compositions may be used in the treatment of clinical conditions for which a GlyT1 inhibitor is indicated such as, for example, schizophrenia. The carrier must be pharmaceutically acceptable to the recipient and must be compatible with, i.e. not have a deleterious effect upon, the other ingredients in the composition. The carrier may be a solid or a liquid and is preferably formulated with at least one compound of formula (I) or a salt or solvate thereof as a unit dose formulation. If desired, other physiologically active ingredients may also be incorporated in the pharmaceutical compositions of the invention.

It will be appreciated by those skilled in the art that the compounds according to the invention may advantageously be used in conjunction with one or more other therapeutic agents, for instance, different antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as atypical antipsychotic drugs and cognitive enhancers.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the invention include for example divalproex, carbamazepine and diazepam.

Suitable atypical antipsychotic drugs which may be used in combination of the compounds of the invention include for example risperidone, olanzapine, ziprasidone, aripiprazole and clozapine.

It will be appreciated that the compounds of the combination or composition may be administered simultaneously (either in the same or different pharmaceutical formulations), separately or sequentially.

The compounds of formula (I) and their pharmaceutically acceptable salts and solvates thereof are also suitable for combination with other typical and atypical antipsychotics to provide improved treatment of psychotic disorders. Particular advantages associated with the combinations, uses and methods of treatment of compounds of formula (I) and their pharmaceutically acceptable salts and solvates thereof include equivalent or improved efficacy at doses of administration which are lower than those commonly used for the individual components. Improved treatments of positive symptoms and/or negative symptoms and/or cognitive symptoms of the psychotic disorder may also be observed. The combinations, uses and methods of treatment of the invention may also provide advantages in treatment of patients who fail to respond adequately or who are resistant to treatment with certain neuroleptic agents.

The combination therapies of the invention are preferably administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the compounds of formula (I) or a pharmaceutically acceptable salt or solvate thereof and at least one neuroleptic agent are within the scope of the current invention. In one embodiment of adjunctive therapeutic administration as described herein, a patient is typically stabilised on a therapeutic administration of one or more of the of the components for a period of time and then receives administration of another component. Within the scope of this invention, it is preferred that the compounds of formula (I) or a pharmaceutically acceptable salt or solvate thereof is administered as adjunctive therapeutic treatment to patients who are receiving administration of at least one neuroleptic agent, but the scope of the invention also includes the adjunctive therapeutic administration of at least one neuroleptic agent to patients who are receiving administration of compounds of formula (I) or a pharmaceutically acceptable salt or solvate thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a further aspect therefore, the invention provides a method of treatment of a psychotic disorder by adjunctive therapeutic administration of compounds of formula (I) or a pharmaceutically acceptable salt or solvate thereof to a patient receiving therapeutic administration of at least one neuroleptic agent. In a further aspect, the invention provides the use of compounds of formula (I) or a pharmaceutically acceptable salt or solvate thereof in the manufacture of a medicament for adjunctive therapeutic administration for the treatment of a psychotic disorder in a patient receiving therapeutic administration of at least one neuroleptic agent. The invention further provides compounds of formula (I) or a pharmaceutically acceptable salt or solvate thereof for use for adjunctive therapeutic administration for the treatment of a psychotic disorder in a patient receiving therapeutic administration of at least one neuroleptic agent.

In a further aspect, the invention provides a method of treatment of a psychotic disorder by adjunctive therapeutic administration of at least one neuroleptic agent to a patient receiving therapeutic administration of compounds of formula (I) or a pharmaceutically acceptable salt or solvate thereof. In a further aspect, the invention provides the use of at least one neuroleptic agent in the manufacture of a medicament for adjunctive therapeutic administration for the treatment of a psychotic disorder in a patient receiving therapeutic administration of compounds of formula (I) or a pharmaceutically acceptable salt or solvate thereof. The invention further provides at least one neuroleptic agent for adjunctive therapeutic administration for the treatment of a psychotic disorder in a patient receiving therapeutic administration of compounds of formula (I) or a pharmaceutically acceptable salt or solvate thereof.

In a further aspect, the invention provides a method of treatment of a psychotic disorder by simultaneous therapeutic administration of compounds of formula (I) or a pharmaceutically acceptable salt or solvate thereof in combination with at least one neuroleptic agent. The invention further provides the use of a combination of compounds of formula (I) or a pharmaceutically acceptable salt or solvate thereof and at least one neuroleptic agent in the manufacture of a medicament for simultaneous therapeutic administration in the treatment of a psychotic disorder. The invention further provides the use of compounds of formula (I) or a pharmaceutically acceptable salt thereof in the manufacture of a medicament for simultaneous therapeutic administration with at least one neuroleptic agent in the treatment of a psychotic disorder. The invention further provides compounds of formula (I) or a pharmaceutically acceptable salt thereof for use for simultaneous therapeutic administration with at least one neuroleptic agent in the treatment of a psychotic disorder. The invention further provides the use of at least one neuroleptc agent in the manufacture of a medicament for simultaneous therapeutic administration with compounds of formula (I) or a pharmaceutically acceptable salt thereof in the treatment of a psychotic disorder.

In further aspects, the invention provides a method of treatment of a psychotic disorder by simultaneous therapeutic administration of a pharmaceutical composition comprising compounds of formula (I) or a pharmaceutically acceptable salt or solvate thereof and at least one mood stabilising or antimanic agent, a pharmaceutical composition comprising compounds of formula (I) or a pharmaceutically acceptable salt or solvate thereof and at least one mood stabilising or antimanic agent, the use of a pharmaceutical composition comprising compounds of formula (I) or a pharmaceutically acceptable salt or solvate thereof and at least one mood stabilising or antimanic agent in the manufacture of a medicament for the treatment of a psychotic disorder, and a pharmaceutical composition comprising compounds of formula (I) or a pharmaceutically acceptable salt or solvate thereof and at least one mood stabilising or antimanic agent for use in the treatment of a psychotic disorder.

In a further aspect, the invention provides a kit-of-parts for use in the treatment of a psychotic disorder comprising a first dosage form comprising compounds of formula (I) or a pharmaceutically acceptable salt or solvate thereof and one or more further dosage forms each comprising a neuroleptic agent for simultaneous therapeutic administration.

Within the context of the present invention, the term psychotic disorder includes those disorders mentioned above, such as schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, dyskinetic disorders, depression, bipolar disorder, cognitive impairment and obsessive-compulsive disorders and all the various forms of the disorders as mentioned herein. which are contemplated as part of the present invention.

Examples of neuroleptic/antipsychotic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benzisothiazolyl-piperazines; triazine such as lamotrigine; dibenzoxazepines, such as loxapine; dihydroindolones, such as molindone; aripiprazole; and derivatives thereof that have antipsychotic activity.

Examples of neuroleptic drugs that are preferred for use in the present invention are shown in Table 1.

TABLE 1
Neuroleptic drugs

| | | | | Dosage | |
|---|---|---|---|---|---|
| Common | | Route of | | Range and | |
| Name | Trade Name | Administration | Form | (Median)^{a} | |
| Clozapine | CLOZARIL | oral | tablets | 12.5-900 | mg/day |
| | | | | (300-900 | mg/day) |
| Olanzapine | ZYPREXA | oral | tablets | 5-25 | mg/day |
| | | | | (10-25 | mg/day) |
| Ziprasidone | GEODON | oral | capsules | 20-80 | mg/twice a day |
| | | | | (80-160 | mg/day) |
| Risperidone | RISPERDAL | oral | solution tablets | 2-16 | mg/day |
| | | | | tablets | |
| | | | | (4-12 | mg/day) |
| Quetiapine | SEROQUEL | oral | tablets | 50-900 | mg/day |
| fumarate | | | | (300-900 | mg/day) |
| Sertindole | SERLECT | | | (4-24 | mg/day) |
| Amisulpride | | | | | |
| Haloperidol | HALDOL | oral | tablets | 1-100 | mg/day |
| | | | | (1-15 | mg/day) |
| Haloperidol | HALDOL | parenteral | injection | | |
| Decanoate | Decanoate | | | | |
| Haloperidol | HALDOL | oral | solution | | |
| lactate | INTENSOL | parenteral | injection | | |
| Chlorpromazine | THORAZINE | rectal | suppositories | 30-800 | mg/day |
| Molindone | MOBAN | | | 50-225 | mg/day |
| | | | | (15-150 | mg/day) |
| Molindone | MOBAN | oral | solution | | |
| hydrochloride | | | | | |
| Loxapine | LOXITANE | | | 20-250 | mg/day |
| | | | | (60-100 | mg/dav) |
| Loxapine | LOXITANE | oral | solution | | |
| hydrochloride | | parenteral | injection | | |
| Loxapine | LOXITANE | oral | capsules | | |
| succinate | | | | | |
| Pimozide | | | | (1-10 | mg/day) |
| Flupenthixol | | | | | |
| Promazine | SPARINE | | | | |
| Triflupromazine | VESPRIN | | | | |
| Chlorprothixene | TARACTAN | | | | |
| Droperidol | INAPSINE | | | | |
| Acetophenazine | TINDAL | | | | |
| Prochlorperazine | COMPAZINE | | | | |
| Methotrimeprazine | NOZINAN | | | | |
| Pipotiazine | PIPOTRIL | | | | |
| Aripiprazole | | | | | |
| Hoperidone | | | | | |

Examples of tradenames and suppliers of selected neuroleptic drugs are as follows clozapine (available under the tradename CLOZARIL®, from Mylan, Zenith Goldline, UDL, Novartis); olanzapine (available under the tradename ZYPREX®, from Lilly ziprasidone (available under the tradename GEODON®, from Pfizer); risperidone (available under the tradename RISPERDAL®, from Janssen); quetiapine fumarate (available under the tradename SEROQUEL®, from AstraZeneca); haloperidol (available under the tradename HALDOL®, from Ortho-McNeil); chlorpromazine (available under the tradename THORAZINE®, from SmithKline Beecham (GSK); fluphenazine (available under the tradename PROLIXIN®, from Apothecon, Copley, Schering, Teva, and American Pharmaceutical Partners, Pasadena); thiothixene (available under the tradename NAVANE®, from Pfizer); trifluoperazine (10-[3-(4-methyl-1-piperazinyl)propyl]-2-(trifluoromethyl)phenothiazine dihydrochloride, available under the tradename STELAZINE®, from Smith Klein Beckman; perphenazine (available under the tradename TRILAFON®; from Schering); thioridazine (available under the trade name MELLARIL®; from Novartis, Roxane, HiTech, Teva, and Alpharma); molindone (available under the tradename MOBAN®, from Endo); and loxapine (available under the tradename LOXITANE®; from Watson). Furthermore, benperidol (Glianimon®), perazine (Taxilan®) or melperone (Eunerpan®)) may be used.

Other preferred neuroleptic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®;), prochlorperazine (available under the tradename COMPAZINE®), methotrimeprazine (available under the tradename NOZINAN®), pipotiazine (available under the tradename PIPOTRIL®), ziprasidone, and hoperidone.

It will be appreciated by those skilled in the art that the compounds according to the invention may advantageously be used in conjunction with one or more other therapeutic agents, for instance, different antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as atypical antipsychotic drugs and cognitive enhancers.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the invention include for example divalproex, carbamazepine and diazepam.

Suitable atypical antipsychotic drugs which which may be used in combination of the compounds of the invention include for example risperidone, olanzapine, ziprasidone, aripiprazole and clozapine.

It will be appreciated that the compounds of the combination or composition may be administered simultaneously (either in the same or different pharmaceutical formulations), separately or sequentially.

Possible formulations include those suitable for oral, sub-lingual, buccal, parenteral (for example, subcutaneous, intramuscular, or intravenous), rectal, topical and intranasal administration and in forms suitable for administration by inhalation or insufflation (either through the mouth or nose). The most suitable means of administration for a particular patient will depend on the nature and severity of the conditions being treated and on the nature of the active compound, but, where possible, oral administration is preferred.

Formulations suitable for oral administration may be provided as discrete units, such as tablets, capsules, cachets, or lozenges, each containing a predetermined amount of the active compound; as powders or granules; as solutions or suspensions in aqueous or non-aqueous liquids; or as oil-in-water or water-in-oil emulsions.

Formulations suitable for sublingual or buccal administration include lozenges comprising the active compound and, typically, a flavoured base, such as sugar and acacia or tragacanth and pastilles comprising the active compound in an inert base, such as gelatin and glycerin or sucrose and acacia.

Formulations suitable for parenteral administration typically comprise sterile aqueous solutions containing a predetermined concentration of the active compound; the solution is preferably isotonic with the blood of the intended recipient. Although such solutions are preferably administered intraveneously, they may also be administered by subcutaneous or intramuscular injection.

Formulations suitable for rectal administration are preferably provided as unit-dose suppositories comprising the active ingredient and one or more solid carriers forming the suppository base, for example, cocoa butter.

Formulations suitable for topical or intranasal application include ointments, creams, lotions, pastes, gels, sprays, aerosols and oils. Suitable carriers for such formulations include petroleum jelly, lanolin, polyethylene glycols, alcohols, and combinations thereof.

The formulations of the invention may be prepared by any suitable method, typically by uniformly and intimately admixing the active compound(s) with liquids or finely divided solid carriers, or both, in the required proportions and then, if necessary, shaping the resulting mixture into the desired shape.

For example, a tablet may be prepared by compressing an intimate mixture comprising a powder or granules of the active ingredient and one or more optional ingredients, such as a binder, lubricant, inert diluent, or surface active dispersing agent, or by moulding an intimate mixture of powdered active ingredient and inert liquid diluent.

Aqueous solutions for parenteral administration are typically prepared by dissolving the active compound in sufficient water to give the desired concentration and then rendering the resulting solution sterile and isotonic.

It will be appreciated that the precise dose administered will depend on the age and condition of the patient and the frequency and route of administration and will be at the ultimate discretion of the attendant physician. The compound may be administered in single or divided doses and may be administered one or more times, for example 1 to 4 times per day.

A proposed dose of the active ingredient for use according to the invention for oral, sub-lingual, parenteral, buccal, rectal, intranasal or topical administration to a human (of approximately 70 kg bodyweight) for the treatment of neurological and neuropsychiatric disorders mediated by a GlyT1 inhibitor, including schizophrenia, may be about 1 to about 1000 mg, preferably about 5 to about 500 mg, more preferably about 10 to about 100 mg of the active ingredient per unit dose which could be administered, for example, 1 to 4 times per day.

The invention is further illustrated by the following non-limiting examples.

### Abbreviations:

### Tetrahydrofuran THF

### Dichloromethane DCM

### Triethylamine TEA

Ethyl acetate EtOAc
Sodium bicarbonate NaHCO₃
Lithium aluminium hydride LAH
Dimethyl sulphoxide DMSO

### DESCRIPTIONS AND EXAMPLES

### Description 1: 4-(2-Fluoro-4-nitrophenyl)morpholine

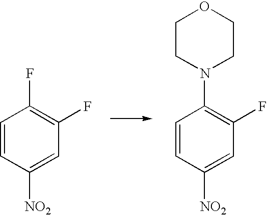

To a solution of morpholine (71.3 ml; 0.82 mol) in THF (60 ml), chilled in an ice-salt bath, was added dropwise 3,4-difluoro nitrobenzene (30 g; 0.18 mol). After the addition, cooling was removed and the reaction mixture warmed to room temperature over 1.25 h. The resulting pale yellow suspension was cooled and aqueous citric acid added dropwise over 25 min, followed by stirring for a further 35 min. Toluene (1 L) was added and the resulting solution washed with water, dried and evaporated to afford the title product (42.6 g; 100%) as a pale yellow solid. ¹H NMR (CDCl₃) δ: 3.29 (4H, m), 3.88 (4H, m), 6.92 (1H, t, J=9 Hz), 7.91 (1H, m), 8.00 (1H, m).

### Description 2: 3-Fluoro-4-(4-morpholinyl)aniline

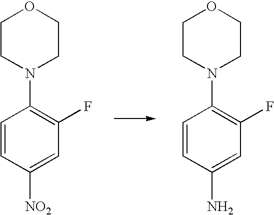

4-(2-Fluoro-4-nitrophenyl)morpholine D1 (42.6 g, 0.19 mol) in ethanol (1.2 L) was hydrogenated over 10% Pd/C paste (4 g) at NTP for 18 h. The resulting mixture was filtered through Kieselguhr and the filtrate evaporated in vacuo to afford the title product (36.9 g, 100%) as a colourless solid. ¹H NMR (CDCl₃) δ: 2.96 (4H, m), 3.55 (2H, br s), 3.84 (4H, m), 6.41 (2H, m), 6.79 (1H, m).

### Description 3: Ethyl 1-[(4-ethylphenyl)carbonyl]-4-piperidinecarboxylate

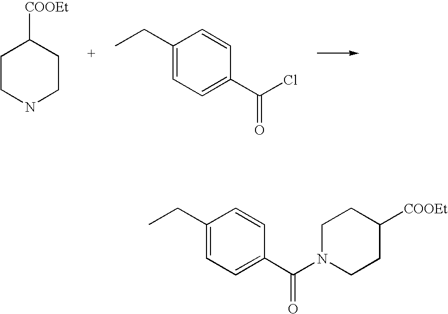

A solution of 4-ethylbenzoyl chloride (21.8 ml, 0.128 mol) in DCM (70 ml) was added dropwise over 30 min. to a stirred solution of ethyl isonipecotate (23.27 g, 0.148 mol) and TEA (41.5 ml, 0.296 mol) in DCM (300 ml) at 0°. The reaction mixture was allowed to warm to room temperature and stirred overnight. The mixture was evaporated and the residue dissolved with EtOAc (350 ml) and washed with water (400 ml), 1M HCl (2×250 ml), NaHCO₃₍ₛₐₜ₎ (250 ml) and brine (2×150 ml). The organic phase was dried over Na₂SO₄, and concentrated, to give the title product as a white solid (40.5 g, 95%). ¹H-NMR (300 MHz, DMSO) δ: 1.1 (6H, m), 1.4 (2H, m), 1.7 (2H, br m), 2.5 (3H, m), 2.9 (2H, br m), 3.45 (1H, br m), 4.0 (2H, q), 4.2 (1H, br m), 7.10 (4H, m).

### Description 4: 1-[(4-Ethylphenyl)carbonyl]-4-piperidinecarboxylic acid

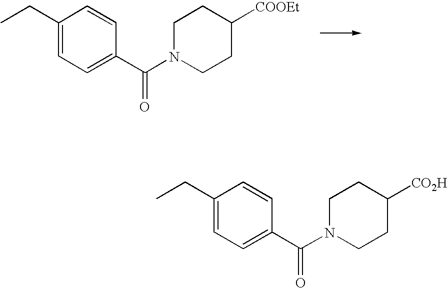

Sodium hydroxide (300 ml 1M) was added with stirring to ethyl 1-[(4-ethylphenyl)carbonyl]-4-piperidinecarboxylate D3 (43.3 g, 0.15 mol) in aqueous methanol (300 ml:300 ml) at room temperature. After 18 h. the mixture was concentrated in vacuo to ca. 500 ml and acidified with 2N HCl to precipitate the acid product as a pale yellow gum which solidified on standing. The solid was filtered and washed with water and dried in vacuo to afford the title product (35.7 g, 91%) as a colourless solid. Mass Spectrum (Electrospray LC/MS): Found 262 (MH⁺). C₁₅H₁₉NO₃ requires 261.

### Description 5: 1-[(4-Ethylphenyl)carbonyl]-N-[3-fluoro-4-(4-morpholinyl)phenyl]-4-piperidinecarboxamide

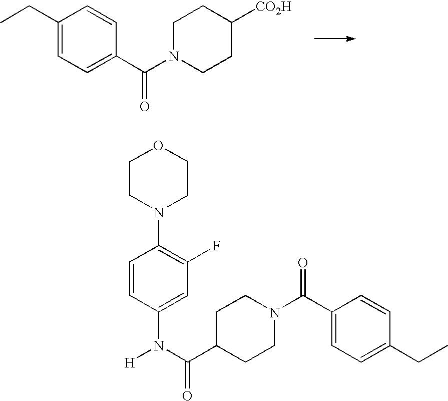

To 1-[(4-ethylphenyl)carbonyl]-4-piperidinecarboxylic acid D4 (12.65 g, 48.4 mmol) in anhydrous toluene (250 ml) under argon at 0° C. was added thionyl chloride (50 ml, 700 mmol) dropwise over 0.5 h. The resulting mixture was allowed to reach room temperature, stirred for 18 h. and then evaporated in vacuo. The residue was re-evaporated from toluene (×2) to afford the crude acid chloride (14 g, 100%) as an amber oil which was used without purification.

A solution of the above acid chloride in DCM (50 ml) was added dropwise to a stirred solution of 3-fluoro-4-(4-morpholinyl)aniline (7.13 g, 36 mmol) and triethylamine (14 ml, 101 mmol) in DCM (450 ml) at 0° C. The resulting mixture was allowed to reach room temperature, stirred for 18 h. and then partitioned with NaHCO₃₍ₛₐₜ₎. The organic was dried and evaporated in vacuo to afford the title product (20.1 g, 91%) as a beige solid. Mass Spectrum (Electrospray LC/MS): Found 440 (MH⁺) C₂₅H₃₀FN₃O₃ requires 439.

### Description 6: N-({1-[(4-Ethylphenyl)methyl]-4-piperidinyl}methyl)-3-fluoro-4-(4-morpholinyl)aniline

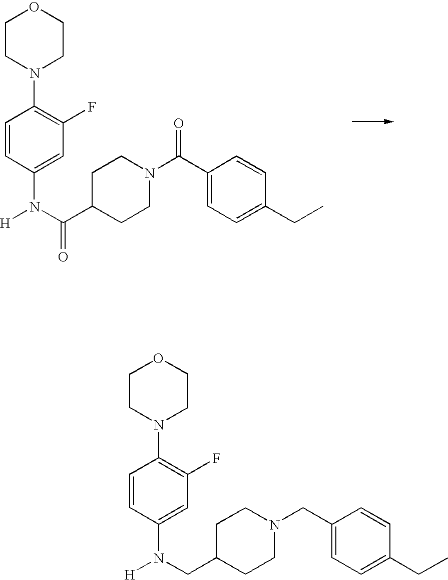

To 1-[(4-ethylphenyl)carbonyl]-N-[3-fluoro-4-(4-morpholinyl)phenyl]-4-piperidine carboxamide D5 (20.1 g, 46 mmol) in anhydrous THF (350 ml) was added LAH (130 ml 1M soln in THF) dropwise under argon at 0° C. The reaction was allowed to warm to room temperature and then heated at reflux for 3 h. After cooling to room temperature, water (14 ml), 2N NaOH (17 ml) and water (14 ml) were added dropwise successively with ice-cooling. After 5 min, sodium sulphate was added and stirring continued for a further 10 min, after which the reaction mixture was filtered. The solid was washed well with THF and the combined filtrate and washings evaporated in vacuo. The residue was chromatographed on silica gel eluting with ethyl acetate to afford the title product (13 g, 94%) as an off-white solid. Mass Spectrum (Electrospray LC/MS): Found 412 (MH⁺). C₂₅H₃₄FN₃O requires 411.

### Example 1

### (2E)-N-({1-[(4-Ethylphenyl)methyl]-4-piperidinyl}methyl)-N-[3-fluoro-4-(4-morpholinyl)phenyl]-3-phenyl-2-propenamide

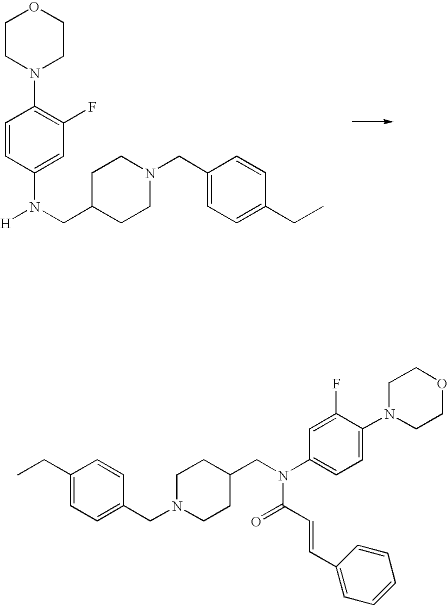

To N-({1-[(4-ethylphenyl)methyl]-4-piperidinyl}methyl)-3-fluoro-4-(4-morpholinyl)aniline D6 (0.075 g; 0.18 mmol) in DCM (1.5 ml) containing TEA (0.075 ml; 0.54 mmol) was added cinnamoyl chloride (0.046 g; 0.27 mmol). After 18 h at ambient temperature the reaction mixture was washed with saturated aqueous NaHCO₃ and the organic phase loaded onto a pre-packed silica gel cartridge which was eluted with DCM and methanol-DCM mixtures to afford the title product (0.084 g; 80%). Mass Spectrum (Electrospray LC/MS): Found 542 (MH⁺). C₃₄H₄₀FN₃O₂ requires 541.

1-18. (canceled)

19. A compound of formula (I) or a salt or solvate thereof: 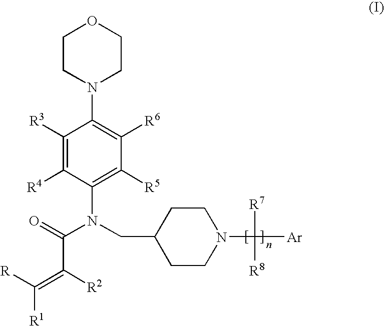
wherein:
R and R¹, together with the carbon atom to which they are attached, form unsubstituted or substituted C₃₋₈cycloalkyl and unsubstituted or substituted C₃₋₈heterocyclyl; or
R is selected from the group consisting of unsubstituted or substituted C₁₋₈alkyl, unsubstituted or substituted C₃₋₈cycloalkyl, unsubstituted or substituted C₃₋₈heterocyclyl, unsubstituted or substituted aryl, and unsubstituted or substituted heteroaryl; and R¹ is selected from the group consisting of hydrogen, halogen, unsubstituted or substituted C₁₋₈alkyl, and unsubstituted or substituted C₃₋₈cycloalkyl;
R² is selected from the group consisting of hydrogen, halogen, unsubstituted or substituted C₁₋₈alkyl and unsubstituted or substituted C₃₋₈cycloalkyl;
R³, R⁴, R⁵ and R⁶ are independently hydrogen and halogen;
R⁷ and R⁸ are independently selected from the group consisting of hydrogen, halogen, and C₁₋₄alkyl, or R⁷ and R⁸ together form C₃₋₄cycloalkyl;
Ar is an unsubstituted or substituted aryl or an unsubstituted or substituted heteroaryl; and
n is 0, 1, 2 or 3.

20. A compound or salt thereof as claimed in claim 19, wherein R is phenyl.

21. A compound or salt thereof as claimed in claim 19, wherein R¹ and R² are both hydrogen.

22. A compound or salt thereof as claimed in in claim 19, wherein R³, R⁴ and R⁵ are hydrogen and R⁶ is fluorine.

23. A compound or salt thereof as claimed in claim 19, wherein phenyl unsubstituted or substituted by one or two groups selected from the group consisting of C₁₋₄alkyl, CF₃, halogen and C₃₋₆cycloalkyl.

24. A compound or salt thereof as claimed in claim 19, wherein n is 1.

25. A compound or salt thereof as claimed in claim 19, wherein R⁷ and R⁸ are both hydrogen.

26. A compound of formula (Ia) or a salt or solvate thereof: 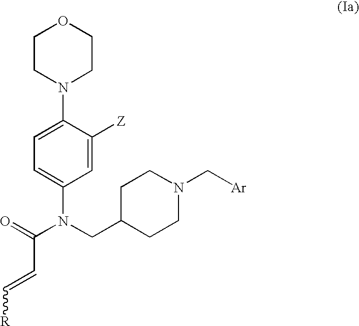
wherein
R is selected from the group consisting of C₁₋₈alkyl, C₃₋₈cycloalkyl, C₃₋₈heterocyclyl, aryl or heteroaryl, each of which is unsubstituted or substituted by one to three groups selected from the group consisting of halogen, oxo, cyano, C₁₋₆alkyl, C₁₋₄alkoxy, haloC₁₋₁₄alkyl, haloC₁₋₁₄alkoxy, arylC₁₋₄alkoxy, C₁₋₄alkylthio, C₁₋₄alkoxyC₁₋₄alkyl, C₃₋₆cycloalkyl, C₃₋₆cycloalkylC₁₋₄alkoxy, C₁₋₄alkanoyl, C₁₋₄alkylsulfonyl, C₁₋₄alkylsulfonylC₁₋₄alkyl, arylsulfonyl, arylsulfonylC₁₋₄alkyl, C₁₋₄alkylamido, C₁₋₄alkylsulfonamidoC₁₋₄alkyl, aroyl, aroylC₁₋₄alkyl, C₁₋₄acyl, aryl, arylC₁₋₄alkyl, C₁₋₄alkylaminoC₁₋₄alkyl, a group R⁹R¹⁰N—, R⁹CON(R¹⁰)(CH₂)ₘ or R⁹R¹⁰NCO(CH₂)ₘ where each of R⁹ and R¹⁰ is independently selected from the group consisting of hydrogen and C₁₋₄alkyl, or R⁹R¹⁰ forms part of a C₃₋₆azacycloalkane ring, and m is 0, 1, 2, 3 or 4;
Z is hydrogen, fluorine or chlorine; and
Ar is phenyl or heteroaryl, each of which is unsubstituted or substituted by one to three groups selected from the group consisting of halogen, oxo, cyano, C₁₋₆alkyl, C₁₋₄alkoxy, haloC₁₋₄alkyl, haloC₁₋₄alkoxy, arylC₁₋₄alkoxy, C₁₋₄alkylthio, C₁₋₄alkoxyC₁₋₄alkyl, C₃₋₆cycloalkyl, C₃₋₆cycloalkylC₁₋₄alkoxy, C₁₋₄alkanoyl, C₁₋₄alkylsulfonyl, C1₋₄alkylsulfonylC₁₋₄alkyl, arylsulfonyl, arylsulfonylC₁₋₄alkyl, C₁₋₄alkylamido, C₁₋₄alkylsulfonamidoC₁₋₄alkyl, aroyl, aroylC₁₋₄alkyl, C₁₋₄acyl, aryl, arylC₁₋₄alkyl, C₁₋₄alkylaminoC₁₋₄alkyl, a group R⁹R¹⁰N—, R⁹CON(R¹⁰)(CH₂)ₘ and R⁹R¹⁰NCO(CH₂)ₘ where each of R⁹ and R¹⁰ is independently selected from hydrogen or C₁₋₄alkyl, or R⁹R¹⁰ forms part of a C₃₋₆azacyloalkane ring and m is 0, 1, 2, 3 or 4.

27. A compound as claimed in claim 19, which is (2E)-N-({1-[(4-ethylphenyl)methyl]-4-piperidinyl}methyl)-N-[3-fluoro-4-(4-morpholinyl)phenyl]-3-phenyl-2-propenamide or a salt or a solvate thereof.

28. A method of preparing a compound as defined in claim 19, comprising the step of:
(a) reacting a compound of formula (II): 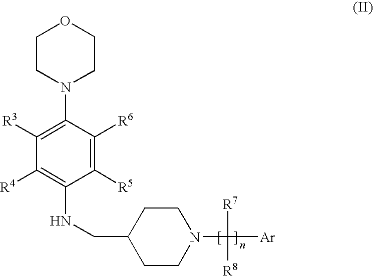
wherein R³ to R⁸, n and Ar are as defined in claim 19 with a compound of formula (III): 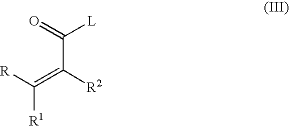
wherein R, R¹ and R² are as defined in claim 19 and L is a leaving group; or
(b) reacting a compound of formula (IV): 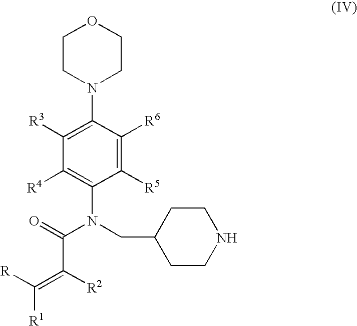
wherein R and R¹ to R⁶ are as defined in claim 19, with a compound of formula (V): 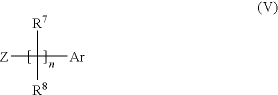
wherein R⁷, R⁸, n and Ar are as defined in claim 19 and Z is a leaving which is halogen, hydroxy or trifluoromethanesulfonyloxy; or
(c) for a compound of formula (I) wherein n is 1, 2 or 3, reacting a compound of formula (IV) as defined above with a compound of formula (VI): 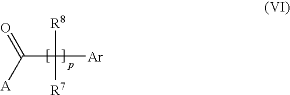
wherein R⁷, R⁸ and Ar are as defined in claim 19, p is n minus one, and A is R⁷ or R⁸;
and then, for step (a), step (b) or step (c):
removing any protecting groups and/or
converting a compound of formula (I) into another compound of formula (I) and/or
forming a salt or solvate.

29. A method of treating a mammal, including a human, suffering from or susceptible to a disorder mediated by GlyT1, which comprises administering an effective amount of a compound or salt thereof as claimed in claim 19.

30. A method as claimed in claim 29, wherein the disorder is schizophrenia, dementia or attention deficit disorder.

31. A pharmaceutical composition comprising a compound or salt thereof as claimed in claim 19, and at least one pharmaceutically acceptable carrier, diluent or excipient.
